(19) United States
(12) Patent Application Publication
(10) Pub. No.: US 2006/0105458 A1
(43) Pub. Date: May 18, 2006

(54) Caged tetracycline (derivatives), their generation, and their use for photoactivated gene expression

(76) Inventors: Sidney Cambridge, Munchen (DE); Beate Curten, Granichen (DE); Tobias Bonhoeffer, Munchen (DE)

Correspondence Address: FULBRIGHT & JAWORSKI L.L.P., 600 CONGRESS AVE., SUITE 2400, AUSTIN, TX, 78701, US

(21) Appl. No.: 10/524,317

(22) PCT Filed: Aug. 4, 2003
(86) PCT No.: PCT/EP03/08616

§ 371 Date: Sep. 30, 2005

(30) Foreign Application Priority Data
Aug. 9, 2002 (EP) 02017953.7

(51) Int. Cl. A61K 31/655; C12N 15/87; C07C 245/06

(52) U.S. Cl. 435/455; 534/653; 514/150

(57) ABSTRACT

Transcriptionally inactivated (caged) tetracycline or tetracycline derivative, wherein the inactivation is caused by reaction of the tetracycline (derivative) with a photosensitive protection compound, said caged tetracycline or tetracycline derivative being capable to be activated again by photolysis.

The present invention relates to novel tetracyclines, methods to prepare these novel tetracyclines, the use of these novel tetracyclines, e.g., in a method to induce gene/transgene expression in a defined set of cells, and optionally in single cells. The present invention further relates to a kit comprising either (i) such new tetracycline and (ii) a gene vector suitable to confer tetracycline-dependent transgene expression (i.e., a vector expressing a fusion protein necessary to induce gene/transgene expression and a vector containing a tetracycline-dependent transgene), or (i) (well known) tetracycline, (ii) a photosensitive protection compound or its precursor, optionally (iii) an oxidizing agent, and (iv) a gene vector suitable to confer tetracycline-dependent transgene expression (i.e., a vectors expressing a fusion protein necessary to induce gene/transgene expression and a vector containing a tetracycline-dependent transgene). The kit may be used to introduce the novel tetracycline and the gene construct, in any temporal order or simultaneously, into target cells, optionally after preparation of the novel tetracycline by reaction of tetracycline and the photosensitive protection compound. Finally, the present invention is directed to a method for the controlled expression of a gene/transgene in a defined set of cells (target cells).

In the last decade or so, several conditional gene expression paradigms in mice have been established and are now enjoying widespread success. Tools that were previously only available in Drosophila, such as the powerful GAL4 system¹, are now being routinely used in mammalian cell culture and tissues, and even more so in the transgenic mouse technology field. Particularly two approaches have gained tremendous popularity: First, using small membrane-permeant molecules to induce ectopic gene expression and second, using site-specific recombinases for post-mitotic mutagenesis in live animals. Cre and FLP recombinases have mainly been employed to irreversibly excise stretches of DNA flanked by specific recognition sites², which in the context of a transgenic knock-in could lead to permanent deletion of the gene or, conversely, lead to permanent expression of the gene if a stop signal had been deleted.

The Tet-System:

The use of small membrane-permeant molecules works as follows: In a two-component system, the first component is a transcription factor (still inactive), whose transcriptional activity is dependent on the binding of the small molecule inducer, while the second component is a construct with the gene of interest being under control of a promoter that is specifically activated upon binding of the (active) transcription factor (which is a complex between the small molecule inducer and the first component, the previously inactive transcription factor). Administration of the small molecule inducer causes a conformational change in the transcription factor, which in turn then increases its affinity for the specific binding sites in the promoter and induces transcription. By far the most prominent system using small molecule inducers is the tetracycline system (tet-system) developed by Bujard and co-workers³. The tet-system is based on a fusion protein (transcription factor, still inactive, but which can be converted into an active transcription factor if complexed with tet, see below) between a mutant form of the tet repressor and a transcriptional activation domain.

Originally, this transcriptional activation domain was VP16 but recently other transcriptional activation domains such as p65 or E2F4 have been employed as well. Binding of tetracycline (tet) or a derivative thereof (as a small molecule inducer, which is a small molecule inducing the conversion of the inactive transcription factor into the active form) to this fusion protein (thereby generating a complex functioning as an active transcription factor) activates transcription of genes under control of the tetracycline promoter. This system is also called the Tet-on system. Alternatively, tetracyclines (derivatives) have been used to de-repress transcription from constitutive promoters. Mainly in plant research, binding of tetracycline (derivatives) to the tet repressor caused un-binding of the repressor-tetracycline complex from the promoter DNA and was thereby used to induce tet-dependent transgenes. Both approaches, the Tet-on system and the use of the original repressor represent cases where gene expression was induced in the presence of tetracycline (derivatives), unlike the Tet-off system where gene expression is induced in the absence of tetracycline (derivatives). The following text focuses on the Tet-on system, however, the invention is also applicable with the Tet-off system and the de-repressed induction with the original repressor.

Currently, one of the most potent tetracycline derivative is doxycycline, which was found to increase gene expression over several magnitudes depending on the system in study. Expression of transgenes was already demonstrated in brain tissues of living mice indicating that tetracycline derivatives such as doxycycline are capable of crossing the blood-brain barrier⁴. In certain instances, induction kinetics was found to be very fast and levels of doxycycline necessary for full transgene expression appear to be non-toxic in almost all systems tested. Clearly, the extent of tet-dependent transgene expression in any given tissue is predetermined by the expression profile of the (inactive) transcription factor. Thus, the researcher is ultimately dependent on the spatial and temporal activity of the promoter used for its expression. Ideally, of course, one would like to control the expression of a transgene and, indeed, of any gene of interest, in any given cell at any given point of time.

Throughout the following text, the alternative expression “gene/transgene” will be frequently used in the context of its induced expression. In the first alternative (gene), the Tet-induced transgene itself is a transcription factor (or any other protein capable of inducing transcription), which in turn—after its introduction into a target cell—activates a ‘normal’ gene (present in the cell) as it always does in the physiological context. In the second alternative, the transgene—under the control of the Tet promoter—is introduced into the target cell where its expression is induced.

Accordingly, the present inventors have posed themselves the object to provide a system suitable to control the expression of a gene/transgene in any given (type of) cell at any time. In order to solve this problem, the present inventors have established a system that allows induction of gene/transgene expression in a space-specific and time-specific manner. That is, a defined set of cells can be induced to express the desired gene, whereas expression in other cells (in cells of other locations) will not be induced.

To this end, the inventors of the present invention developed a photoactivatable version of the inducible tetracycline (tet) system described above. The inventors developed “caged” tetracycline and tetracycline derivatives such as doxycycline and anhydro-tetracycline. These caged molecules are reversibly inactivated with a photosensitive protection moiety (synonymous: caging moiety) by reaction with a photosensitive protection compound (synonymous: caging compound). Reversible inactivation means that the caged molecules are no longer capable to function as a small molecule inducer as described above in the Tet-system³ and to convert the inactive transcription factor into its active form. However, photolysis of the caged tetracycline or its derivative (e.g., by means of UV light) regenerates transcriptionally active tetracycline or tetracycline derivatives (capable to render the previously inactive transcription factor active).

Derivatives of tetracycline according to the present invention are defined to include the following compounds: chlorotetracycline, oxytetracycline, demethylchlorotetracycline, doxycycline monohydrate, minocycline, metacycline, anhydro-tetracycline, and rolitetracycline. Furthermore, according to the present invention synthetic molecules that mimic the properties of naturally occurring tetracyclines will also be considered tetracycline derivatives.

Induction of gene/transgene expression in defined cells is a valuable tool for biomedical research. On the other hand, the present inventors have been capable to demonstrate that the doses of, e.g., UV light necessary for uncaging the tet compound are non-toxic (cells in irradiated areas did not show signs of necrosis even several days after the experiment).

Thus, one aspect of the present invention relates to transcriptionally inactivated (caged) tetracycline or tetracycline derivatives, wherein the inactivation is caused by reaction of the tetracycline (derivative) with a photosensitive protection compound, said caged tetracycline or tetracycline derivative being capable to be activated again by photolysis. The photosensitive protection compound is a compound exhibiting a moiety, preferably an aromatic or heteroaromatic, more preferably a rigid aromatic or heteroaromatic moiety, suitable to absorb light in the UV range of 10 to 450 nm, in particular 200 to 450 nm. Suitable groups meeting with such criterion are indolinyl, benzyl, coumarinyl, desoxybenzoinyl, or hydroxyphenacyl. Of course, it may be preferable that the aromatic residue be additionally substituted with a chromophor (capable to cause a shift of the wavelength of the light absorbed by the (hetero)aromatic compound into the direction of UV/VIS, e.g., to longer wavelengths). Suitable examples of chromophors are alkoxy groups, preferably methoxy and ethoxy, amino groups such as —NH₂, —NHR₁, and —NR₁R₂, wherein R₁ and R₂ are independently an alkyl, aryl, alkaryl, or aralkyl group, and the nitro group (—NO₂). In case of nitrobenzyl compounds, it is preferred that the —NO₂ group is located in ortho (2-nitrobenzyl). Preferred photosensitive protection compounds are compounds comprising a 2-nitrobenzyl, an α-carboxy-2-nitrobenzyl (CNB), a nitroindolinyl, a 7-methoxycoumarinyl, and a 1-(4,5-dimethoxy-2-nitrophenyl) ethyl (DMNPE) residue.

The photosensitive protection compound further exhibits a reactive group. This reactive group is destined to react with the functional group of the tetracycline (derivative), thereby caging the latter. Accordingly, alkyl hydrazone residues (preferably, alkyl is a cyclic or linear C1 to C6 alkyl, with the ethyl residue being most preferred) and oxiranyl residues (C₂R₄O, wherein all four R's may be identical or different, and may be H or alkyl; preferably, alkyl is a linear C1 to C4 alkyl, with the methyl residue being most preferred) are suitable reactive groups. As discussed in more detail below (see section Synthesis of Caged Tetracyclines), alkyl hydrazone residues will not be reacted with the functional group of tetracycline (derivative) but after a conversion into the respective diazoalkyl residues. Quite conversely, oxiranyl compounds exhibit sufficient reactivity to be reacted with tetracycline (derivative) directly. Accordingly, alkyl hydrazone-containing compounds should be correctly termed photosensitive protection precursor compounds, whereas diazoalkyl as well as oxiranyl and 1,2-dihydroxyethyl compounds are photosensitive protection compounds within the meaning and definition according to the present invention.

According to a preferred embodiment of this first aspect of the present invention, the tetracycline derivative is doxycycline, anhydro-tetracycline, or minocycline. According to another preferred embodiment of this aspect, said tetracycline or tetracycline derivative contains a functional group such as an amino, an amide, a carbonyl, a sulfhydryl, or preferably a hydroxy function (e.g., doxycycline exhibits a hydroxy at the 3, 5, 10, 12, 12a-position).

A further aspect of the present invention relates to a method to prepare the transcriptionally inactivated (caged) tetracycline or tetracycline derivative as defined above, the method comprising the step of reacting a (photosensitive protection) compound comprising a group (residue) capable to react with the functional group of the tetracycline (derivative) and a group capable to absorb electromagnetic radiation in the UV range as defined above (e.g., the aromatic and heterooaromatic residues indolinyl, benzyl, coumarinyl, desoxybenzoinyl, or hydroxyphenacyl) with tetracycline or a derivative thereof.

According to a preferred embodiment of this aspect of the present invention, the functional group of the tetracycline (derivative) is an amino, an amide, a carbonyl, a sulfhydryl, or a hydroxy group, and the reactive group of the compound is a diazoalkyl or oxiranyl/1,2-dihydroxyethyl group.

According to still another preferred embodiment of this aspect, the method is performed by reaction of either doxycycline, anhydro-tetracycline, or minocycline with 1-(1-diazoethyl)-4,5-dimethoxy-2-nitrobenzene or with 1-diazoethyl-7-methoxycoumarin. According to still another preferred embodiment, the photosensitive protection precursor compound is converted into the photosensitive protection compound by using manganese dioxide as an oxidizing agent.

Still a further aspect of the present invention is the use of the transcriptionally inactivated (caged) tetracycline or tetracycline derivative as defined above to induce expression of a gene/transgene either at a defined point of time or in a defined set of cells (in a limited type/number of cells; that is, dependent on the position of the cells, some are induced and others are not), or both. Still a further aspect of the present invention is a kit, comprising, in a suitable container means, (i) either a transcriptionally inactivated (caged) tetracycline (derivative) or a transcriptionally active (uncaged) tetracycline (derivative), a photosensitive protection compound or its precursor and, optionally, an oxidizing agent; and (ii) a gene vector suitable to confer tetracycline-dependent transgene expression (i.e., a vector expressing a fusion protein necessary to induce gene/transgene expression and a vector containing a tetracycline-dependent transgene.

The term fusion protein as used herein relates to transcription factors, in particular the transcription factors described above in the context of the Tet-on and Tet-off system.

The kit may be used to introduce the novel tetracycline and the gene construct, respectively, in any temporal order or simultaneously, into target cells, optionally after preparation of a caged tetracycline by reaction of tetracycline and the photosensitive protection compound.

A still further aspect of the present invention is directed to an in vitro- and in vivo-method for the controlled expression of a tetracycline-dependent gene/transgene in a defined set of cells (target cells), the method comprising the following steps:
(a) reacting tetracycline or a tetracycline derivative with a photosensitive protection compound to prepare a caged tetracycline or a caged tetracycline derivative;
(b) introducing, in any order of the steps including simultaneously, said caged tetracycline or tetracycline derivative, the tetracycline-dependent transgene, and the gene encoding a transcription factor that is inactive absent its binding to tet or a tet derivative into said defined set of cells (target cells), wherein introduction of the transgene and the gene for the transcription factor in its inactive form is not required, provided the target cells express the transgene and the transcription factor gene, respectively; and
(c) irradiating the defined set of cells.

According to a preferred embodiment, irradiation of the cells is performed by irradiation with UV light or occurs via 2-photon or multi-photon microscopy. Another preferred embodiment of the method of the present invention relates to a method further comprising step (d) of detecting the polypeptide (protein) expressed in the defined set of cells.

“Caging” basically means that a tetracycline (derivative) is derivatized with a photosensitive protection compound via a functional group on the tetracycline (derivative), such as amino, amide, carbonyl, sulfhydryl, or preferably hydroxy, and a reactive group located on the photosensitive protection compound.

Photosensitive protection compounds inactivate biologically active molecules by disrupting the interaction of functional groups with other functional groups on the same or on another molecule. This disruption can occur because caging introduces a more or less bulky residue, which sterically inhibits the interaction due to its size or because caging changes the normal physical-chemical properties of the functional group, which inhibits the normal interaction of this group with other partners. For example, caging can transform an ionic lysine residue into a non-polar group so that the normal electrostatic interaction with other, negatively charged residues is blocked. Photosensitive protection compounds are therefore ‘function-blocking molecules’ and contain a reactive group, which can react with residues of the tetracycline (derivative). Preferred function-blocking molecules have been mentioned above (e.g., aromatic/heteroaromatic, in particular benzyl, indolinyl, etc. compounds).

Reaction of the photosensitive protection compound with a functional group of the tetracycline (derivative) that is necessary for its transcriptional activity (e.g., the hydroxy groups in position 3, 10, 12, and 12a, or, for example, the carbonyl groups in position 1 and 11) inhibits its activity as a small molecule inducer (as defined above and ⁵) and its capacity to induce transcription by forming a functional complex (active transcription factor) with the fusion protein. Depending on the functional group of the tetracycline molecule to be employed, a suitable reactive group of the photosensitive protection compound (e.g., a diazoethyl-substituted compound) is required to enable tetracycline (derivative) and photosensitive protection compound to specifically react with each other, thereby rendering the small molecule inducer (tetracycline or derivative) transcriptionally (reversibly) inactive.

To give an example for a suitable combination: the hydroxy group of tetracycline or one of its derivatives such as doxycycline, identified in Examples 2 and 3 below, is reacted with a diazoethyl group of a photosensitive protection compound formed after conversion by oxidation of an ethyl hydrazone residue (of a photosensitive protection precursor compound, see the compounds prepared in Examples 1 and 3c) into said diazoethyl group, as described in Examples 2 and 3d.

Photolysis of caged tetracycline (derivatives), for instance by UV-irradiation, releases the free tetracycline (derivative), which in turn associates with the (inactive) transcription factor, that is, e.g., the fusion protein as defined above, or with any other (inactive) transcription factor (capable (i) to be activated by binding to tetracycline or a derivative thereof and, once activated, (ii) to induce gene expression under control of the tet promoter) to generate the active transcription factor, thereby inducing expression of a gene/transgene.

Preferred genes/transgenes are tumor suppressor genes such as the retinoblastoma (Rb) and p53 genes, genes encoding (i) transcription factors such as engrailed and Pax6, (ii) signaling molecules such as sonic hedgehog, (iii) growth factors, and (iv) members of the Wnt family as well as their receptors, or genes implicated in diseases such as presenilins (Alzheimer) and alpha-synuclein (Parkinson's).

For the purpose of providing a positive control, other preferred genes are the CAT (chloramphenicol acetyl transferase) gene, the GFP (green fluorescence protein) gene, the EGFP (enhanced green fluorescence protein) gene, or any other easily detectable reporter gene. Additionally, for the purpose of developing a model to study tumorigenesis, other preferred genes are oncogenes such as the ras, the myc, the jun/fos, the myb, or the abl gene.

In principle, by a local application of UV-light or any other suitable means inducing photolysis (e.g., 2-photon microscopy) of the caged compound, exquisite spatial gene induction can be obtained simply by modulating the width of the beam (gene expression is induced only in irradiated areas). Photoactivated gene expression would therefore be a very powerful tool for conditional mutagenesis.

According to the present invention, photolysis of the caged tetracycline (derivative) and induction of gene/transgene expression is accomplished by, e.g., irradiation with UV light or 2-photon or multi-photon microscopy. In fact, in terms of practical aspects, irradiation with UV light is clearly preferred as an embodiment of the present invention because it is simpler and more straightforward. On the other hand, 2-photon and multi-photon microscopy are preferred in terms of the effect(s) conferred: Photolysis by 2-photon and multi-photon microscopy is less toxic to cells and tissues than is UV irradiation. Additionally, it can penetrate deeper into tissue (e.g., 2-photon microscopy uncages tetracycline or its derivative with two photons instead of one as in uncaging with UV light). Thus, one may consider 2-photon microscopy simply as a different, albeit more complex, light source. Because each photon in 2-photon microscopy/photolysis has only about half the energy (that is why two photons are needed) their wavelength is about twice as long according to the equation: energy×wavelength=constant. Photons with longer wavelengths in the (near) infrared region of >700 nm interact less with biological tissue and can therefore penetrate deeper into tissue as the photons get less absorbed/scattered.

2- (and also multi-) photon microscopy is particularly important for uncaging in dense tissues such as the brain. Here, two near-infrared photons, instead of one in the UV/visible range, excite the chromophore, which only occurs if there is a very high density of photons in an exceedingly small focal volume of about 1 μm³. In highly scattering brain tissue, 2-photon microscopy allows resolution of individual spines within a depth of up to 500 μm⁶. Clearly, 2-photon microscopy provides the most suitable technology for analysis. The caging compound used for modification of doxycycline displays a good 2-photon cross-section so that photoactivated gene expression is also possible with a 2- or multi-photon set-up. In animals or thick tissues, light scattering precludes conventional one-photon approaches as an optical mean to study or manipulate biological processes. This problem has been overcome by 2- and multi-photon microscopy.

Synthesis of Caged Tetracyclines:

A preferred compound to cage tetracyclines is 1-(1-diazoethyl)-4,5-dimethoxy-2-nitrobenzene which is prepared from 1-(4,5-dimethoxy-2-nitrophenyl)-ethanone hydrazone (obtainable according to the method described in Example 1) by oxidation with MnO₂. Thus, the actual caging compound according to this preferred embodiment is the diazoethyl compound. However, as this compound (as are diazo compounds in general) is unstable, it is preferred to prepare it freshly (directly before tetracycline or its derivative is to be caged) from 1-(4,5-dimethoxy-2-nitrophenyl)-ethanone hydrazone by oxidation by means of a suitable oxidizing agent (which is separated from the reaction mixture prior to the caging reaction). A preferred oxidation agent is MnO₂.

1-(1-diazoethyl)-4,5-dimethoxy-2-nitrobenzene or 1-diazoethyl-7-methoxycoumarin, if reacted with any of the hydroxy groups in position 3, 10, 12, and 12a, or, with any of the carbonyl groups in position 1 and 11 of tet or any of its derivatives, couples to the tet compound via an ether bridge which has previously been shown to display good photolysis kinetics and good photolysis efficiency⁷.

It is preferred that the caging compound has a methyl group attached on the carbon of the, e.g., benzyl function (that the actual caging compound is a diazoethane rather than a diazomethane) so that photolysis produces a ketone and not an aldehyde reaction side product. Ketones are less reactive and therefore less toxic than aldehydes, which helps to minimize undesired side effects of the photoactivation reaction.

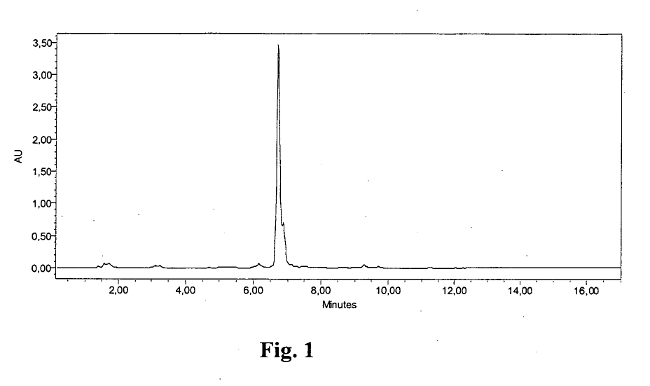

Caged tetracyclines are purified to homogeneity, as determined by HPLC analysis. When doxycycline is the tetracycline to be caged, and DMNPE is the caging moiety, HPLC analysis reveals a major (95%) and a minor (5%) peak, both of them representing DMNPE-caged doxycycline analogs (see FIG. 1) as determined by mass spectroscopy.

Due to the more hydrophobic nature of the caging compound, caged tetracyclines such as caged doxycycline is less soluble in water compared to the unmodified tetracycline/doxycycline but solubility is still high enough to prepare a stock solution in normal buffers (molar concentration sufficiently high to achieve a final concentration of 0.1 to 1,000 μM, preferably 0.15 to 500 μM, 0.25 to 250 μM, 0.5 to 100 μM, 0.75 to 50 μM, 1 to 25 μM, 2 to 15 μM, or most preferably 5 to 10 μM). When stored at −20° C., a stock solution of 10 to 200 mM DMNPE-caged tetracycline/doxycycline in DMSO is stable for at least a year. If diluted down to the final concentration of 0.1 to 1 μM, DMNPE-caged tetracycline/doxycycline displays good stability even under incubation conditions (only about 10% of the molecules spontaneously decompose within 24 h at 37° C.).

According to a preferred embodiment of the present invention, the caged molecule is doxycycline caged by 1-(4,5-dimethoxy-2-nitrophenyl)-ethyl (DMNPE). According to a further preferred embodiment UV irradiation is confined to a small area, entailing that the transcriptionally active tetracycline, especially doxycycline, is released locally only, and gene expression is induced with unprecedented spatial (and optionally temporal) resolution. In a particularly preferred embodiment, gene expression is induced in distinct (single) cells.

Using DMNPE-caged doxycycline (that is, doxycycline caged after reaction with, e.g., 1-(1-diazoethyl)-4,5-dimethoxy-2-nitrobenzene), the inventors could demonstrate specific, time-dependent, and local expression of genes/transgenes (highly spatiotemporal resolution of gene expression is possible employing any caged tet or tet derivative) in irradiated CHO cells or organotypic hippocampal Müller cultures.

The significance of such a tool for basic biomedical research is enormous. Potential applications include, but are not limited to, cell lineage tracing during development, localized pre- or post-synaptic induction of specific genes to study the contribution to synaptic plasticity, or induction of oncogenes in individual cells in a wild-type background to study the development of spontaneous tumorigenesis.

FIG. 1 depicts the HPLC diagram of DMNPE-caged doxycycline. The compound has a purity of >95%.

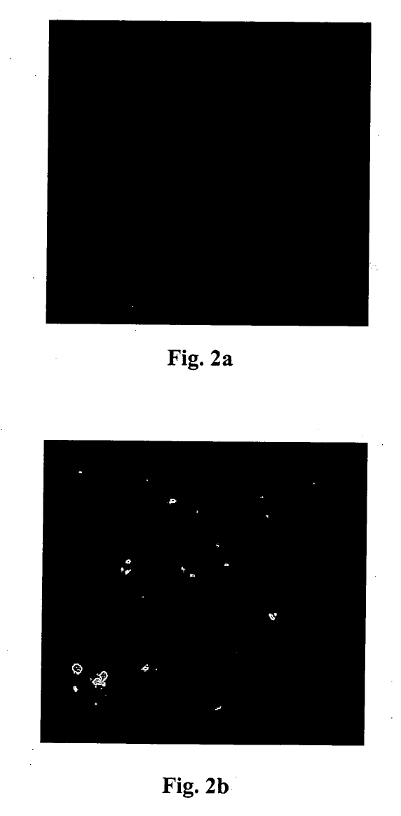

FIG. 2 illustrates the result of an experiment to assess the transcriptional activity of DMNPE-caged doxycycline. In this experiment, CHO cells doubly transfected and expressing the fusion protein (inactive transcription factor) and containing a tetracycline-dependent EGFP (Enhanced Green Fluorescent Protein) construct were used as a simple cell culture assay. In fact, irradiation of cells that were incubated with DMNPE-caged doxycycline displayed widespread EGFP fluorescence (FIG. 2a), while cells in the same dish that were not irradiated did not fluoresce (FIG. 2b).

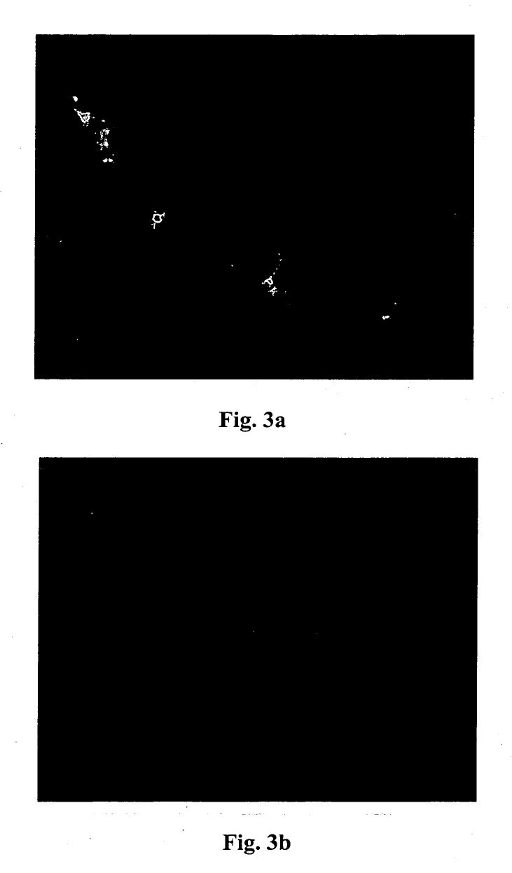

FIG. 3 depicts a second assay to test photoactivation in tissues, exemplified by the use of organotypic hippocampal Müller cultures. Two adenoviruses (a gift from G. Akusjärvi), one constitutively expressing the (inactive) transcription factor and one containing a tet-dependent chloramphenicol transferase (CAT) construct as a transgene, were used to infect wild-type rat or mouse brain slices. After administration of caged doxycycline to the cells by adding it to the medium and subsequent photoactivation, CAT expression was assessed immuno-histochemically. FIG. 3a depicts CAT expression in a few cells after a single pulse of UV irradiation while the unirradiated control slice (FIG. 3b) did not display any CAT expression. CAT expression in the irradiated slice is mainly seen in cells at the border of the slice where cells become infected with viruses much more rapidly than in the center. Nevertheless, this assay clearly shows that photoactivated gene expression is also possible in tissues.

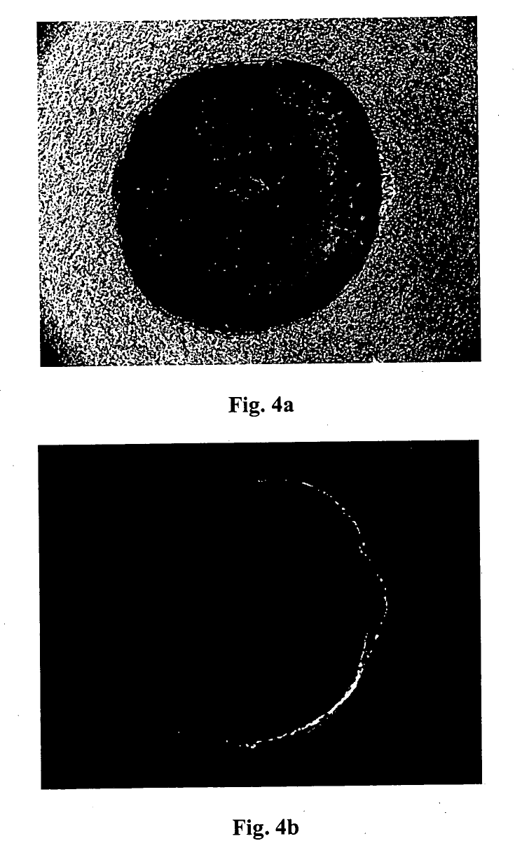

FIG. 4 illustrates the result of a further experiment performed by the present inventors. That is, in order to test if photoactivated gene expression can be used to induce gene expression in a locally restricted manner, adenovirus-infected Müller cultures were irradiated only in one half of the slice. FIG. 4a shows a darkfield image of the entire slice while the fluorescence image of FIG. 4b depicts CAT staining only in the irradiated, right half of the culture. Again, fluorescence is mostly seen at the slice boundary where the virus most readily infects cells of the culture. Thus, photoactivated gene expression provides the researcher with excellent spatial control over gene expression.

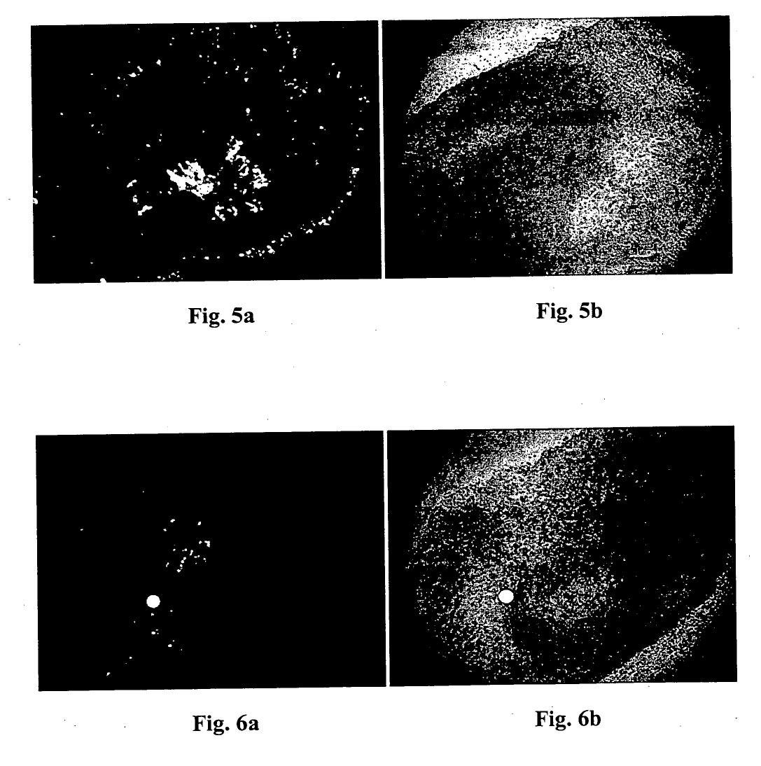

FIG. 5 depicts a modified assay to test photoactivation in tissues, exemplified again by the use of organotypic hippocampal Müller cultures. An adenovirus containing a tet-dependent GFP construct as a transgene (purchased from University of Iowa, Vector Core), and the above described adenovirus that constitutively expresses the (inactive) transcription factor rtTA were used to infect wild-type rat or mouse brain slices. To determine the maximal level of GFP expression that is possible with this approach, unmodified doxycycline was added in saturating concentrations (2 μM). Following incubation overnight, a subpopulation of cells at the edge as well as cells in the center of the culture displayed strong GFP expression (FIG. 5a). The bright-field image on the right allows normal visualization of the culture (FIG. 5b).

FIG. 6: In a new set of experiments, photoactivation was done by using thin optical fibers (200 μm) for irradiation with the UV light coming from a Xenon arc lamp. The UV beam could be positioned with the help of a recording electrode that was aligned to be in the center of the circular irradiation area. About half of the culture was irradiated with this arrangement. The position of the electrode is indicated by the red dot. The culture was incubated with 1.5 μM caged doxycycline one hour prior to irradiation. Cultures were irradiated three times for 2 min with an interval of 1 hour. FIG. 6a shows GFP positive cells that are within the circular irradiation area. FIG. 6b indicates the electrode position relative to the whole culture.

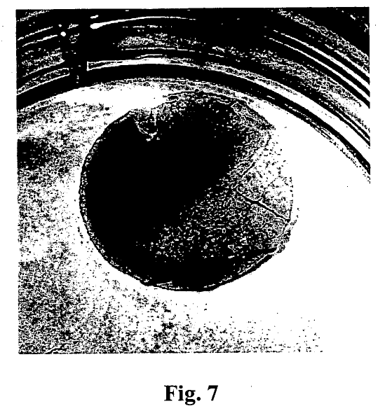

FIG. 7 demonstrates that photoactivated gene expression also works in plants. For this, genetically modified tobacco plants were used, that activate a reporter enzyme, beta-glucuronidase (GUS) in the presence of doxycycline⁸. A small circular piece of such a tobacco leaf was incubated in 2 μM caged doxycycline and was subsequently partially irradiated with a hand-held UV lamp. In the irradiated half, significant GUS expression was seen while a sharp boundary separates the unstained, unirradiated area from it. This strongly suggests that photoactivated gene expression affords a very high spatial resolution for the induction of transgenes.

The advantages of the method according to the present invention, i.e., of using a caged tetracycline (derivative) such as a caged doxycycline are obvious: Any of the other conditional gene expression methods do not give the researcher the option of being able to freely choose the population of target cells in which the gene of interest is to be expressed. Likewise, being able to predetermine the time of expression is usually not possible. In fact, compared to the ‘normal’ Tet-system, photoactivated gene expression has the additional advantage of rapid activation as the slow induction kinetics of the Tet-system is probably due to slow diffusion of the doxycycline molecules. With caged doxycycline, the tissue can be incubated without any effect until saturation is reached so that the induction kinetics solely depends on the process of transcription and translation.

Many different research applications of photoactivated gene expression are possible. Some potential in vivo and in vitro applications include the following: (1) cell lineage tracing during development, (2) pre- or postsynaptic induction of genes/transgenes to study their contribution to synaptic plasticity, and (3) sporadic induction of oncogenes in a background of normal, wild-type tissue to study spontaneous tumorigenesis.
(1) Cell lineage tracing: Induction of a permanent marker via Cre or FLP recombinase will allow to follow the trajectory and final maturation of any particular cell. This approach may become especially important if birthplace/birthtime determine cell fate. With single cell resolution, a correlation between the origin of the cell and its final fate will be more easily identified.
(2) Pre- or postsynaptic induction: In studies of synaptic transmission, a central question remains the pre- or postsynaptic manifestation of synaptic plasticity. Until recently, the contribution of any particular gene to synaptic plasticity was usually determined by assessing the effects of a null-mutant. While this approach was very successful, little has been learned about the actual site of action, i.e. in most cases it remained unclear whether it is a pre- or postsynaptic effect of the protein in study. With photoactivated gene expression, genes can be manipulated either presynaptically or postsynaptically so that specific, local contributions can be separated. One of the main reasons for establishing photoactivated gene expression in brain tissue is that the inherent complexity of this organ requires new tools that can help to unravel its intricate processes. To understand such intricate processes one needs the proper controls. However, a common problem in the neurosciences is that controls often have to be done in other samples, for example measuring LTP in mutant versus wild-type slices. With the spatial resolution afforded by photoactivated gene expression, the actual experiment, such as a photoactivated local gene knock-out, and the unirradiated control can be in the same sample. Thus, this approach overcomes the inter-sample variation.

(3) Sporadic induction of oncogenes: One of the hallmarks of spontaneous tumorigenesis is the conversion of a normal, healthy cell into a metastatic cell. Currently, very few models exist that can recapitulate this conversion of a single cell in a wild-type background of surrounding cells. Therefore, photoactivation of oncogenes in individual cells should be a very powerful model of tumorigenesis.

Clearly, many other research applications exist where caged doxycycline can be employed for photoactivated gene expression. For example, researchers have used the normal Tet-system for studies of neurodegnerative diseases such as transmissible spongiform encephalopathy (prion disease) or Huntington's disease. Again, photoactivated gene expression may be a valuable tool for disease models due to its high spatiotemporal resolution of gene expression.

The present invention is further illustrated by the following non-limiting examples.

### EXAMPLES

### Example 1

### Synthesis of 1-(4,5-dimethoxy-2-nitrophenyl) ethanone hydrazone

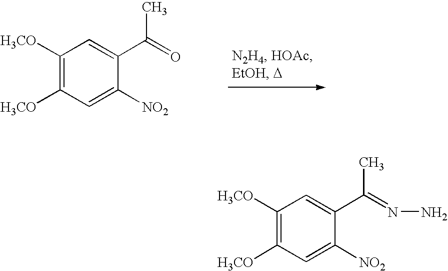

1-(4,5-Dimethoxy-2-nitrophenyl) ethanone, which is prepared according to literature⁷ procedures, is suspended in ethanol. Glacial acetic acid (1 equiv.) and hydrazine monohydrate (2 equiv.) are added and the mixture is heated to 40-80° C. for 1-4 h. After cooling to ambient temperature, the reaction mixture is diluted with water and extracted several times with chloroform. The organic layers are combined, extracted with water, dried over sodium sulfate, filtered and concentrated to dryness. The oily residue is crystallized from ether.

### Example 2

### Synthesis of 1-(4,5-dimethoxy-2-nitrophenyl)-ethyl-caged doxycycline

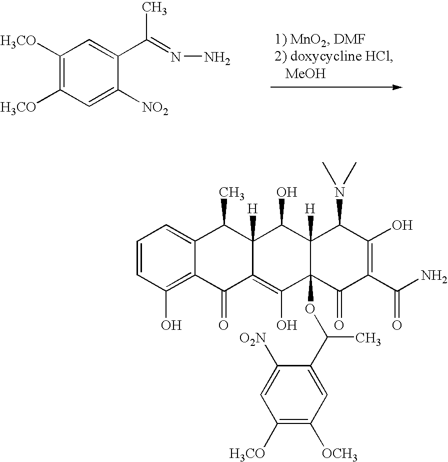

1-(4,5-dimethoxy-2-nitrophenyl) ethanone hydrazone is suspended in DMF and briefly treated with excess manganese dioxide. The suspension is filtered over celite. The filter cake is rinsed with dimethylformamide (DMF). The filtrate and the washings are combined and added dropwise to a solution of doxycycline hydrochloride (1 equiv.=0.21 mmol) in 0.1 M methanolic potassium hydroxide (1 equiv.). The reaction mixture is stirred at ambient temperature and under protection from light for 18-36 h. The reaction mixture is concentrated and the crude product is purified by preparative HPLC. The fractions containing the corresponding mass (654 amu) are collected and lyophilized.

¹H-NMR (DMSO): 15.22 (s), 11.48 (s), 10.47 (s), 9.71 (s), 7.66-7.64 (s), 7.57-7.52 (t), 7.48 (s), 7.15 (s), 6.95-6.88 (dd), 6.48-6.43 (q), 5.65 (s), 3.97 (s), 4.16 (s), 3.92-3.87 (m), 3.49-3.41 (q), 3.49 (d), 2.71-2.64 (m), 2.55-2.52 (m), 1.72 (d), 1.46 (d).

### Example 3

### Synthesis of 1-(7-methoxycoumarin-4-yl)-ethyl-caged doxycycline

a) Synthesis of 4-(1-hydroxyethyl)-7-methoxycoumarin

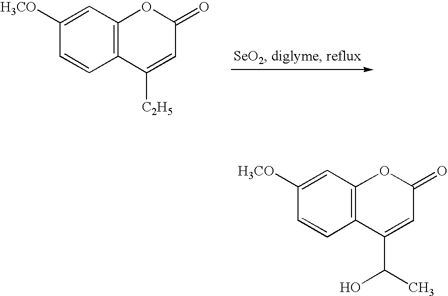

Commercially available 4-ethyl-7-methoxycoumarin and selenium dioxide (1.4 equiv.) are suspended in diglyme and heated to 100-140° C. for 2 to 8 h. Upon cooling, the selenium is filtered off and rinsed with ethanol. The filtrate and the washings are combined and the solvent is evaporated. The residue is purified by column separation on silica gel.

¹H-NMR (acetone-d₆): 7.78-7.75 (m, 1 H), 6.93-6.90 (m, 1 H), 6.90-6.89 (m, 1 H), 6.39 (d, 1 H), 5.24-5.21 (m_{c}, 1 H), 4.66-4.64 (m, 1 H), 3.92 (s, 3 H), 1.51 (d, 3 H).

b) Synthesis of 4-acetyl-7-methoxycoumarin

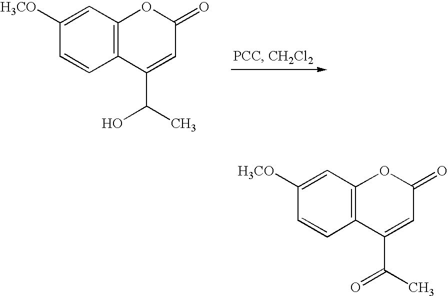

The 4-(1-hydroxyethyl)-7-methoxycoumarin and pyridinium chlorochromate (PCC) on alumina are suspended in methylene chloride and stirred at ambient temperature for 10-20 h. The supernatant is filtered and the dark residue rinsed with methylene chloride twice. The solution and the washings are combined and chromatographed over silica gel yielding the desired product as light yellow needles upon evaporation of the solvent.

¹H-NMR (acetone-d₆): 7.86-7.83 (m, 1 H), 6.95-6.93 (m, 2 H), 6.67 (s, 1 H), 3.93 (s, 3 H), 2.67 (s, 3 H)

c) Synthesis of 4-(1-ethanehydrazonoyl)-7-methoxycoumarin

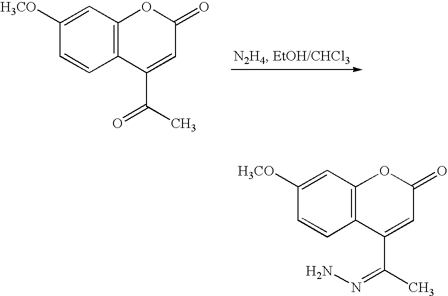

4-Acetyl-7-methoxycoumarin is dissolved in a 3:1 mixture of ethanol and chloroform. After addition of hydrazine monohydrate (1.3 equiv.) the reaction mixture is stirred at ambient temperature for at least 12 h. The solvent is evaporated and the residue treated with 1 ml ethanol. The white precipitate is filtered and dried.

¹H-NMR (CDCl₃): 8.06-8.03 (m, 1 H), 6.87-6.81 (m, 2 H), 6.21 (s, 1 H), 5.76 (s, br, 2 H), 3.87 (s, 3 H), 2.15 (s, 3 H).

d) Synthesis of 1-(7-methoxycoumarin-4-yl) ethyl-caged doxycycline

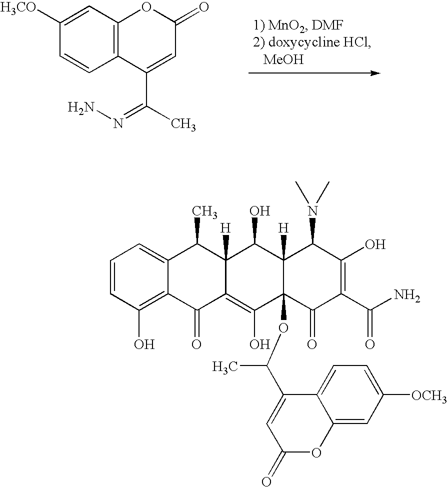

4-(1-Ethanehydrazonoyl)-7-methoxycoumarin is dissolved in DMF and treated with manganese dioxide for 1-20 min. After filtration over celite, the solution is added dropwise to a solution of doxycycline hydrochloride in methanol. The mixture is stirred at ambient temperature and protection from light for 5-24 h. The reaction mixture is diluted with 10 ml methanol, filtered and the filtrate concentrated to 0.5-1 ml residual volume. The solution of the crude material is purified by HPLC, the fraction containing the corresponding mass (664 amu) is lyophilized.

### Example 4

### Photoactivation of CHO Cells

CHO cells mentioned above (that is, cells expressing the fusion protein³ and containing a tetracycline-dependent EGFP) were used as a simple cell culture assay were propagated and plated according to standard cell culture procedures in Ham's F-12 medium. Cells were plated in 35 mm dishes with a glass bottom from Mattek. Two days after plating, cells were overlaid with 1% low-gelling agarose in Ham's F-12 containing 1 mM Na-butyrate and 0.3 μM caged doxycycline. Subsequently, a subset of cells was locally irradiated with a hand-held 6W UV lamp (365 nm) for 30 sec. Following overnight incubation, EGFP expression was assessed with a fluorescence microscope.

### Example 5

### Photoactivation of Müller Cultures

Mouse or rat hippocampal Müller cultures were prepared according to standard procedures from P5-7 animals. After incubation at 37° C. for one week, the medium was inoculated with two different adenoviruses: one constitutively expressing the (inactive) transcription factor³, and the other containing a tet-dependent CAT gene as a transgene. Following overnight incubation, 0.15 μM caged doxycycline was added to the medium, incubated for 1 h, and slices or selected regions of slices were irradiated for 10 sec with an upright fluorescence microscope using an appropriate filter (XF3000, OMEGA OPTICAL). Following overnight incubation, CAT fluorescence was detected immunohistochemically with a polyclonal anti-CAT antibody (Sigma).

### REFERENCES

(1) A. H. Brand and N. Perrimon (1993). Development 118, 401-415
(2) M. Lewandoski (2001). Nat. Rev. Genet. 2, 743-755
(3) M. Gossen et al. (1995). Science 268, 1766-1769
(4) M. T. Hasan et al. (2001). Genesis 29, 116-122
(5) S. R. Adams and R. Y. Tsien (1993). Annu. Rev. Physiol. 55, 755-784
(6) K. Svoboda et al. (1997). Nature 385, p 161-165
(7) M. Wilcox et al. (1990). J. Org. Chem. 55, 1585-1589
(8) C. Gatz et al. (1992). The Plant Journal 2, 397-404

1. Transcriptionally inactivated (caged) tetracycline or tetracycline derivative, wherein the inactivation is caused by reaction of the tetracycline (derivative) with a photosensitive protection compound, said caged tetracycline or tetracycline derivative being capable to be activated again by photolysis.

2. Transcriptionally inactivated (caged) tetracycline or tetracycline derivative according to claim 1, wherein said derivative is doxycycline, anhydro-tetracycline, or minocycline.

3. Transcriptionally inactivated (caged) tetracycline or tetracycline derivative according to claim 1, wherein said tetracycline or tetracycline derivative contains a functional group such as an amino, an amide, a carbonyl, a sulfhydryl, or preferably a hydroxy function.

4. Transcriptionally inactivated (caged) tetracycline or tetracycline derivative according to claim 1, wherein said photosensitive protection compound is a compound comprising an α-carboxy-2-nitrobenzyl (CNB), a coumarinyl, a desoxybenzoinyl, or a hydroxyphenacyl residue.

5. Transcriptionally inactivated (caged) tetracycline or tetracycline derivative according to claim 1, wherein said photosensitive protection compound is 1-(1-diazoethyl)-4,5-dimethoxy-2-nitrobenzene or 4-(1-diazoethyl)-7-methoxycoumarin.

6. Method to prepare the transcriptionally inactivated (caged) tetracycline or tetracycline derivative as defined in any of the preceding claims, the method comprising the step of reacting a compound comprising a reactive group capable to react with the functional group of the tetracycline (derivative) and a group capable to absorb electromagnetic radiation in the UV range with tetracycline or a derivative thereof.

7. The method according to claim 6, wherein the functional group of the tetracycline (derivative) is an amino, an amide, a carbonyl, a sulfhydryl, or a hydroxy group, and wherein the reactive group of the compound is an alkyl hydrazone/diazoalkyl, an oxiranyl, or a 1,2-dihydroxyethyl group.

8. The method according to claim 6, wherein either of doxycycline, anhydro-tetracycline, or minocycline is reacted with 1-(1-diazoethyl)-4,5-dimethoxy-2-nitrobenzene or 4-(1-diazoethyl)-7-methoxycoumarin.

9. The method according to any of claim 6, wherein the photosensitive protection compound is obtained by oxidation of a photosensitive protection precursor compound with manganese dioxide as an oxidizing agent.

10. Use of the transcriptionally inactivated (caged) tetracycline or tetracycline derivative as defined in any of claim 1 to induce expression of a gene/transgene either at a defined point of time or in a defined set of cells, or both.

11. Kit, comprising, in a suitable container means,
(i) either a transcriptionally inactivated (caged) tetracycline (derivative) or a transcriptionally active (uncaged) tetracycline (derivative), a photosensitive protection compound or its precursor and, optionally, an oxidizing agent; and
(ii) a gene vector suitable to confer tetracycline-dependent transgene expression (i.e., a vector expressing a fusion protein necessary to induce gene/transgene expression and a vector containing a tetracycline-dependent transgene)

12. In vitro- and in vivo-method for the controlled expression of a tetracycline-dependent gene/transgene in a defined set of cells (target cells), the method comprising the following steps:
(a) reacting tetracycline or a tetracycline derivative with a photosensitive protection compound to prepare a caged tetracycline or a caged tetracycline derivative, both as defined in 1;
(b) introducing, in any order of the steps, said caged tetracycline or tetracycline derivative, the tetracycline-dependent transgene, and the gene encoding a transcription factor that is inactive absent its binding to tet or a tet derivative into said defined set of cells (target cells), wherein introduction of the transgene and the gene for the transcription factor in its inactive form is not required, provided the target cells express the transgene and the transcription factor gene, respectively; and
(c) irrradiating the defined set of cells.

13. The method of claim 12, wherein the cells are irradiated with UV light.

14. The method of claim 12, wherein irradiation of the cells occurs via 2- or multi-photon microscopy.

15. The method of claim 12, the method further comprising the following step of (d) detecting the polypeptide (protein) expressed in the defined set of cells.
